(19) United States
(12) Patent Application Publication
(10) Pub. No.: US 2006/0166283 A1
(43) Pub. Date: Jul. 27, 2006

(54) Means for detecting neurodegenerative processes and applications of same

(76) Inventors: Andre Delacourte, Faches-Thumesnil (FR); Nicolas Sergeant, Ronchin (FR); Annick Wattez, Villeneuve D'Ascq (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/526,899

(22) PCT Filed: Sep. 8, 2003
(86) PCT No.: PCT/FR03/02667

§ 371 Date: Sep. 21, 2005

(30) Foreign Application Priority Data
Sep. 6, 2002 (FR) 02 11 100

(51) Int. Cl. G01N 33/567; C12N 9/22; C07K 16/40

(52) U.S. Cl. 435/7.2; 435/199; 530/388.26

(57) ABSTRACT

The invention relates to novel markers of the neurodegenerative process, comprising the α chain of the modified ATP synthase. The invention also relates to methods of detection, diagnosis, antibodies, kits and applications of same.

A subject of the invention is means for detecting neurodegenerative processes and their applications.

It relates more particularly to novel markers of the neurodegenerative process, methods of detection and diagnosis, as well as their therapeutic and diagnostic uses.

Alzheimrer's disease (AD) is a neurodegenerative disease which leads to the loss of intellectual functions and therefore to the progressive and irreversible onset of dementia. The future of this disease presents a challenge with the ageing of the population as age is the major risk factor.

99% of the forms observed do not run in families. For the forms which run in families, pathological mutations are observed on the APP gene on chromosome 21, and on the PS1 and PS2 (preseniline 1 and 2) genes which are found on chromosomes 14 and 1.

Two degenerative processes characterize Alzhemer's disease. Amyloidogenesis, which results from 2 dysfunction of the APP protein, and neurofibrillary degeneration (NFD), which corresponds to accumulation of tau protein in the nerve cells.

Dysfunction of the APP protein is at the very origin of Alzheimer's pathology, but it is the extension of neurofibrillary degeneration in the cerebral territories which is better correlated with the expression of clinical manifestations. 10 stages of neurofibrillary degeneration invasion have been determined. The relation between amyloid deposits and neurofibrillary degeneration is still little known, but tau pathology is Certainly potentialized by dysfunction of the APP protein.

Numerous hypotheses have been formulated in order to explain neuronal degeneration and death. It seems undeniable that a number of factors are involved, including the loss of function of the APP protein, the neurotoxicicity of the amyloid peptide, age, neuronal repair phenomena, modulated by apolipoprotein E, co-morbidity, immunological and inflammatory reactions, as well as oxidative stress.

Numerous works suggest that oxidation could contribute either directly or indirectly to neuronal death in Alzheimer's disease. However, there is no direct molecular argument to support this hypothesis.

The invention is based on the demonstration of a deleterious mechanism which is associated with the process of neurofibrillary degeneration observed in the development of Alzheimer's disease and related pathologies.

In particular, it relates to the direct demonstration of the involvement of a molecule of the mitochondrial respiratory chain and in particular of a complex V protein of this respiratory chain. It concerns a particular location of the protein and an insolubilization of the ATP synthase a chain which then accumulates in the degenerating neurons and is closely associated with the tau protein or its polymers (pathological filaments in neurofibrillary degeneration) observed in particular in Alzheimer's disease.

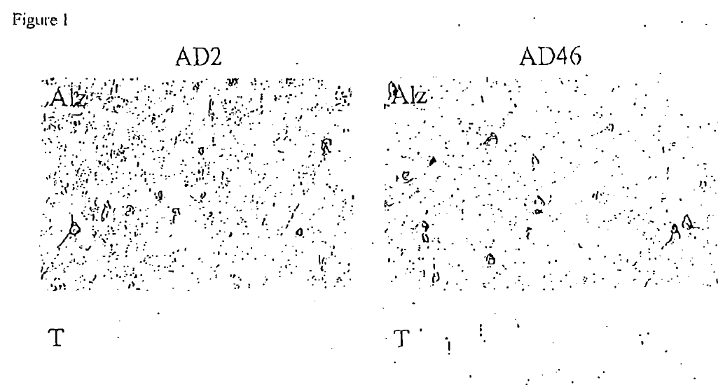

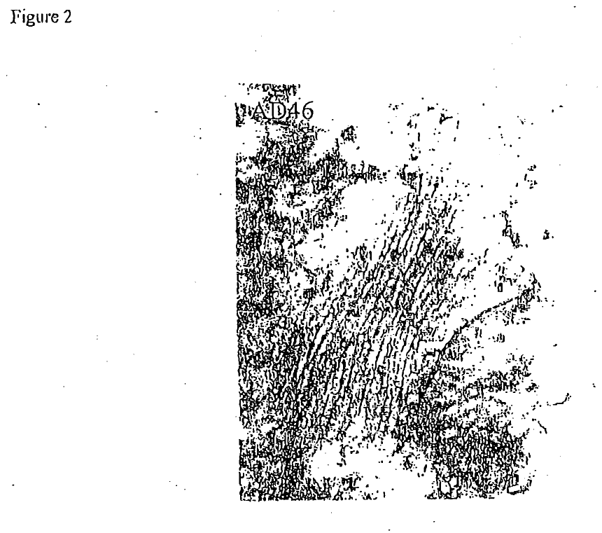

Research carried out by the inventors has led them to develop a monoclonal antibody, named AD46, which detects the pathological filaments which accumulate in the degenerating neurons as Alzheimer's disease Progresses. This antibody recognizes the pathological fibrillary cluster of NFD at the optical microscope scale and the PHF (paired helical filaments) at the electron microscope scale (FIGS. 1 and 2). The immunomarking is collocated with the aggregated tau proteins, which are the basic constituents of NFD. However, the AD46 marking sometimes precedes the tau immunomarking of neurofibrillary degeneration, revealed by phospho-dependent and phospho-independent anti-tau antibodies. The marking of the AD46 antibody being independent of the tau marking, it demonstrates the existence of a third aggregative process, the essential component of which is the human ATP synthase a chain. The marking with the AD46 antibody sometimes occurring before the marking of the tau proteins also demonstrates that it is an early marker of the neurodegenerative process.

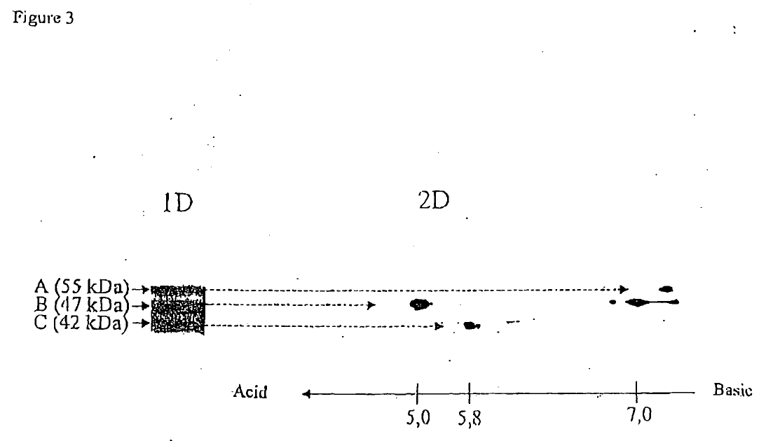

The one and two-dimensional immunodot technique reveals that the AD46 antibody recognizes one major protein and two minor proteins in a homogenate of human cerebral tissue (FIG. 3). These three proteins are different from tau proteins and catabolism products of tau proteins. In fact, AD46 does not recognize normal and pathological tall proteins, extracted from homogenates of cerebral tissue from Alzheimer's patients and control subjects, nor recombinant tau proteins.

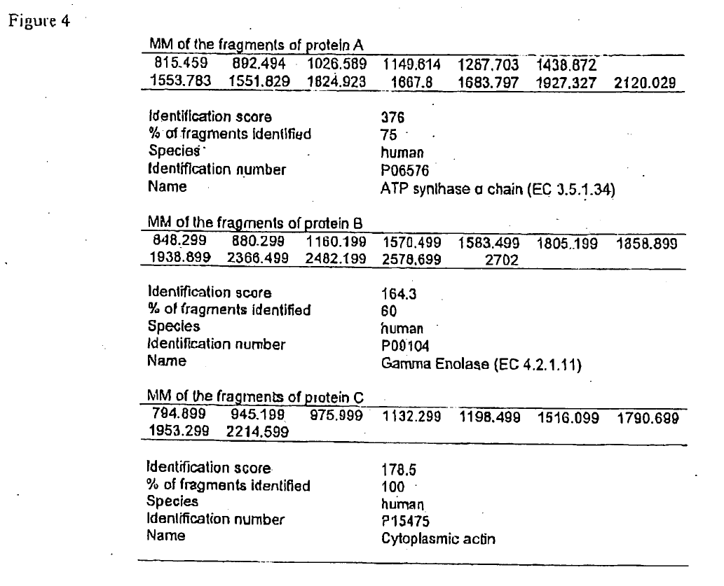

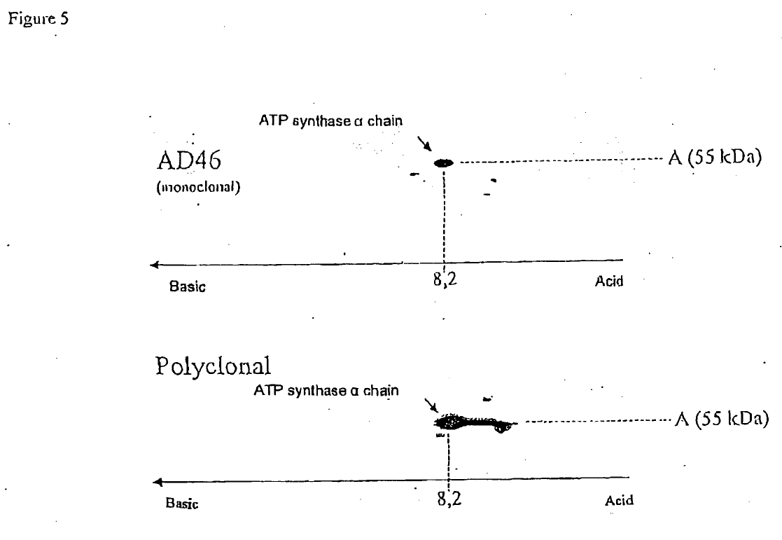

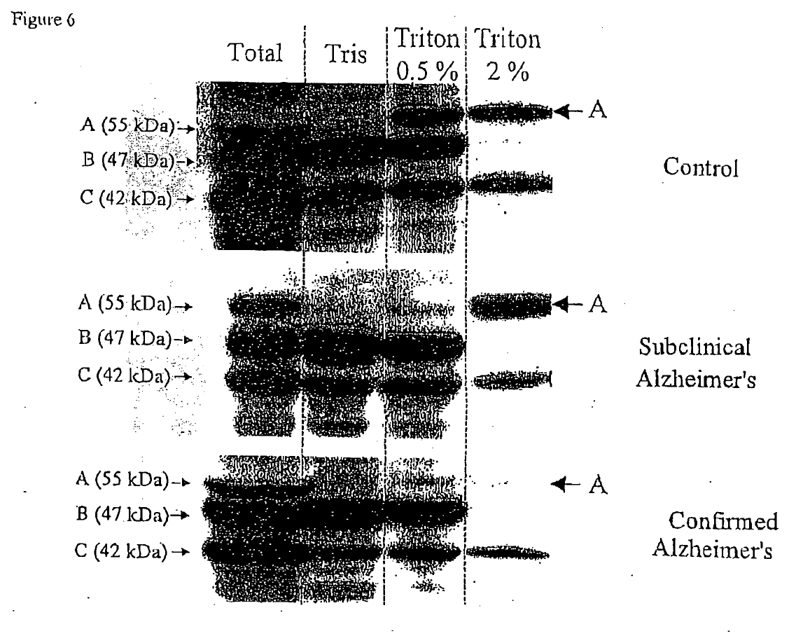

Two-dimensional analysis and a proteomic approach have made it possible to identify the major protein and the two minor proteins detected by AD46. The molecular masses are 42, 47 and 55 kDa (FIG. 1). After 2D electrophoresis, the respective isoelectric points of each polypeptide are 5.0, 5.9, 7.0 and 8.2 (FIG. 3). Characterization by mass spectrometry demonstrated that the 42 and 47 kDa polypeptides correspond respectively to cytoplasmic actin, gamma-enolase and alpha-enolase. The 55 kDa major antigen with a pI of 8.2 corresponds to the ATP synthase α chain (FIGS. 4 and 5). This result for the α chain is confirmed by the use of a polyclonal antibody directed against this protein (FIG. 6).

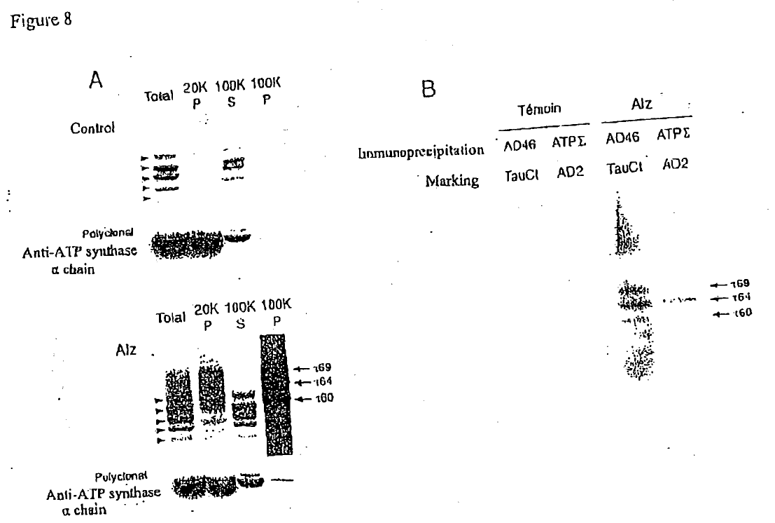

The ATP synthase α chain is a novel constituent of NFD, to the extent that a polyclonal antibody directed against the recombinant ATP synthase α chain recognizes human neurons in NFD (FIG. 8), at an optical and electron microscope scale.

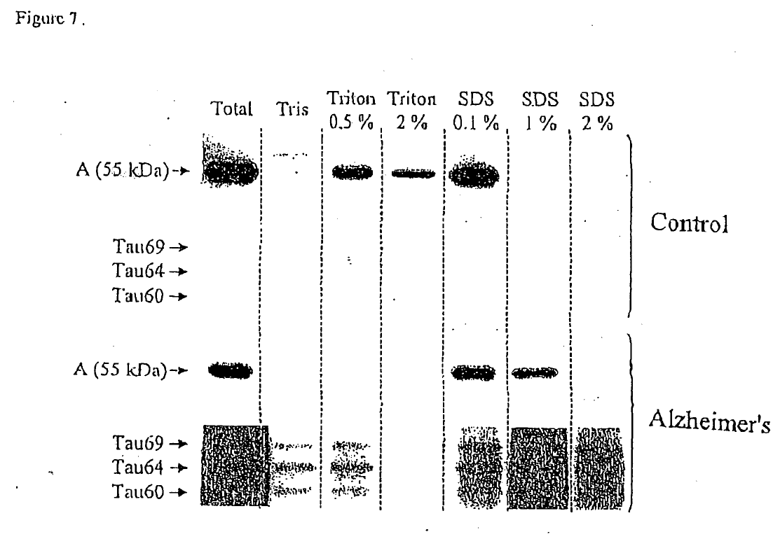

The ATP synthase α chain is not associated indirectly and secondarily with NFD, but linked directly with the degenerative process. In fact, a study of the distribution of the ATP synthase α chain in the different protein fractions of cerebral tissue of control subjects and Alzheimer's patients demonstrated that the disappearance of the ATP synthase α chain occurs progressively during the onset of the NFD process (FIGS. 6 and 7). Whereas the ATP synthase α chain (FIGS. 6 and 7, component A) detected by the monoclonal antibody AD46 is observed essentially in the 0.5% and 2% Triton fractions of cerebral homogenates of control subjects, it is absent from the 0.5% Triton fraction of cerebral homogenates of Alzheimer's patients at the subclinical stage and absent from the 0.5% and 2% Triton fractions of the protein fractions of homogenates of Alzheimer's patients (FIG. 6). In the same manner, the use of the polyclonal antibody directed against the ATP synthase a chain also shows a move from the solubility of this protein into more insoluble fractions (0.1 and 1% SDS fractions, FIG. 7). The insolubilization of the ATP synthase α chain follows the aggregation of the tau proteins, as shown by the co-markings of the ATP synthase α chain/tau (FIG. 7). This disappearance of the ATP synthase α chain is observed specifically in the cerebral regions affected by the neurofibrillary degeneration of patients at the subclinical stage (Stages 4 to 7 of Delacourte et al., 1999) (FIG. 6). It is observed in all the cerebral regions of the patients at an advanced stage of the disease (Stages 8 to 10 of Delacourte et al., 1999) (FIGS. 6 and 7). Moreover, there is co-immunoprecipitation of the aggregated tau proteins and the ATP synthase α chain and vice-versa, demonstrating the direct involvement of the ATP synthase α chain in the aggregative process of the tau proteins (FIG. 8). Purification of the aggregated tau proteins also makes it possible to co-purify the ATP synthase α chain (FIG. 8) the biological function of the ATP synthase α chain indicates that the process of neurofibrillary degeneration in the development of Alzheimer's disease is linked, at least in part, to a singular location and an accumulation of the protein in the cytoplasm of the neurons in neurofibrillary degeneration. Indirectly, the location of the ATP synthase α chain could alter the oxide-phosphorylation process of the mitochondrial respiratory chain, or initiate a degeneration process.

The ATP synthase α chain is also associated with other biological functions. It could be a cytokine membrane receptor, such as angiostatin or polypeptide II activator of the endothelial cells and monocytes. It is also described in several cell compartments, such as the plasma membrane, the peroxisomes. It could also be 2 chaperone protein. The association of the ATP synthase α chain with tau protein aggregates could alter one or more of the biological functions of the ATP synthase α chain.

The invention therefore relates to novel markers of the neurodegenerative process, constituted by the ATP synthase a chain having undergone pathological modifications resulting from said process.

It relates more particularly to the use of the ATP synthase α chain having undergone pathological modifications resulting from a neurodegenerative process as a marker of neurodegenerative diseases, including the tauopathies.

In particular, these markers are characterized in that the modifications of the ATP synthase α chain are of functional, location, structural and/or antigenic type. These markers are particularly suitable for the detection of the neurodegenerative process of any pathology with a process of neurofibrillary degeneration and aggregation of the tau protein, more particularly, the neurodegenerative process of Alzheimer's disease.

The functional, location, structural modifications of the ATP synthase α chain presented by the markers according to the invention can be in particular, respectively, its insolubility, its location in the cytoplasm of the cell and/or the formation of aggregates at the level of the cerebrum. These modifications can also be of conformational and/or post-translational type, including maturation.

By “location in the cytoplasm”, the intra-ribosomal location in particular is excluded.

The formation of aggregate relates to the ATP synthase a chain alone, or in interaction with the tau proteins, in order to induce tauopathy, i.e. the aggregation of the tau proteins.

The discovery of these novel markers, linked directly with the degenerative process of Alzheimer's disease, opens up diagnostic and therapeutic perspectives of great interest.

The invention therefore also relates to a method of detection and/or diagnosis in vitro of the neurodegenerative process, characterized in that one of the markers according to the invention is detected in a sample to be analyzed. The method advantageously comprises the use of sets of antibodies directed against the normal protein and/or against modifications of the ATP synthase α chain.

The method according to the invention is particularly suitable for detection of the degenerative process of klzheimer's disease.

The techniques used in such a method vary: immuno-chemical detection, in particular by 1D and/or 2D electrophoresis coupled with an immunodot, development by polyclonal antibodies or monoclonal antibodies directed against the ATP synthase α chain, immuno-assay and/or radioimmuno-assay.

The capture of the pathological ATP synthase α chain can be followed by complementary analysis by mass spectrometry: immunocapture of the pathological ATP synthase α chain is then carried out and the products captured by mass spectrometry are analyzed.

Conversely, and complementarily, it is possible to carry out the immunocapture of the pathological tau proteins then to look for and identify all or part of the pathological ATP synthase α chain associated with the tau proteins, by mass spectrometry.

Any type of samples to be analyzed can be used: neuronal tissues or cells, non-neuronal tissues or cells, in particular biological liquids, preferably blood.

The diagnostic method according to the invention can moreover comprise a stage of evaluation of the degree of the pathology by establishing an index based on the relationship between the normal level of ATP synthase α chains in control subjects in a defined protean fraction, with respect to the level observed at the advanced stage of Alzheimer's disease.

Alternatively, the extent of the pathology can be evaluated by establishing an index based on modifications of the ATP synthase α chain in a patient compared with a control subject.

Detection of functional and/or location and/or structural and/or antigenic modifications of the ATP synthase α chain in the peripheral biological liquids and/or in the cerebral tissue and/or in peripheral tissue makes it possible to carry out an early diagnostic test. In particular, the neuronal death which occurs during Alzheimer's disease causes a release of tau proteins into the cerebrospinal liquid (CSL). The presence of the ATP synthase α chain in the biological liquids, the association of which with the tau proteins has been demonstrated can therefore complete the biological diagnosis in an advantageous manner. The invention allows a novel diagnostic approach to the neurodegenerative pathologies, in particular Alzheimer's disease. The ATP synthase α chain being observed early, sometimes before the aggregation of the tau proteins, its detection in the biological liquids, and in particular the CSL, is a marker of choice, which can be used for the purposes of early diagnosis or determination of risk factor.

The invention also proposes a method for evaluating the pathological interaction of the ATP synthase α chain with the tau proteins, and reciprocally, using appropriate techniques such as for example bio-sensor, ELISA, immunoprecipitations, mass spectrometry. This method makes it possible to define an index of pathological transformation of the ATP synthase α chain and/or the tau protein. It also relates to kits for implementing said method of evaluation.

The methods of detection, diagnosis or evaluation according to the invention can advantageously be used in order to establish an ante and post-mortem diagnosis of the neurodegenerative diseases, in particular Alzheimer's disease, at the subclinical stage (“mild cognitive impairment”) at the clinical stage, in order to carry out pharmacological screening and therapeutic tests on molecules effective against the neurodegenerative pathologies, in particular of tauopathy or Alzheimer's disease type, and/or in order to establish and validate cell models and/or animal models of neurodegenerative pathologies, in particular of tauopathy or Alzheimer's disease type.

The invention also covers any animal or cell model, characterized in that it expresses an ATP synthase α chain having a maturation signal defect or a post-translational modification linked with the degenerative process.

It relates, for example, to (animal or cell) models expressing the ATP synthase α chain in the cytoplasm of cells, i.e. by eliminating the first 47 amino acids which constitute the maturation signal of the ATP synthase α chain for its integration with complex V of the mitochondrial respiratory chain. These models should thus reproduce the accumulation of the ATP synthase α chain in the cytoplasm.

It relates in particular to research into the displacement of the ATP synthase a chair in a reconstituted system, called a cybrid (Sheehan et al., 1997). This system consists of evaluating the functions of the mitochondria obtained from blood platelets of patients suffering from Alzheimer's disease in a reconstituted cell system. The orientation of the ATP synthase α chain in this system can be evaluated and an orientation index defined (the value 100 corresponding to normal subjects, the value 0 to patients suffering from Alzheimer's disease).

The invention also relates to the therapeutic use, to the extent that the ATP synthase α chain is a precise pharmacological target, which can be modulated by the action of specific inhibitors and activators, the protein itself and/or its ligands, and/or the complex V of the mitochondrial respiratory chain in general. In fact, its action relates directly to the process of neurofibrillary degeneration, and can also relate to the APP metabolism.

The use of a kit for detection of the ATP synthase a chain, for the diagnosis of neurodegenerative diseases, in particular for the detection of Alzheimer's disease, also falls within the scope or the invention.

The invention finally proposes a diagnostic kit comprising sets of polyclonal and/or monoclonal antibodies directed against patterns of pathological conformation of the ATP synthase α chain resulting from a neurodegenerative process. It also covers said antibodies. Advantageously, said kit contains reagents making it possible to carry out an immunochemical assay, in particular of ELISA, immunodot, Western blots, dots-blots, radioimmuno-assay or immuno-assay type. Complementary assay by mass spectrometry is also possible, in order to unambiguously detect alterations of the pathological ATP synthase α chain.

Example 3 hereafter also proposes a method for the preparation of immunological tools against the ATP synthase a chain comprising the following stages:
selective extraction of neurofibrillary degeneration from human tissue affected by a neurodegenerative process,
extraction of the pathological ATP synthase α chain,
use of the purified ATP synthase α chain as antigen for the production of polyclonal antibodies.
   For the production of monoclonal antibodies, the clones selected preferably possess the following immunological properties:
Detection of the Alzheimer's type neurodegenerative process by immunohistochemical study
Detection of the pathological ATP synthase α chain
Co-immunoprecipitation of the pathological tau proteins

Other characteristics and advantages of the invention will become apparent in the examples which follow, with references to FIGS. 1 to 11:

FIGS. 1 and 2 illustrate the immunodetection of Alzheimer's type neurofibrillary degeneration by the AD46 antibody, at an optical and electron microscopy scale.

FIGS. 3, 4 and 5 relate to the characterization of the proteins detected by AD46, by one-dimensional electrophoresis (FIG. 3; 1D), then by two-dimensional electrophoresis (FIG. 3; 2D), by mass spectrometry analysis of the spot detected by AD46 (FIG. 4), and finally by 2D gel comparison of the immunodetection of AD46 with respect to an antibody directed against the identified protein, namely the ATP synthase a chain (FIG. 5).

FIGS. 6 and 7 show the insolubilization of the ATP synthase α chain; the latter disappears from the Triton fractions as Alzheimer's develops.

FIG. 8 illustrates the interaction of the ATP synthase α chain with the tau protein; it is extracted and immuno-precipitated with the tau protein.

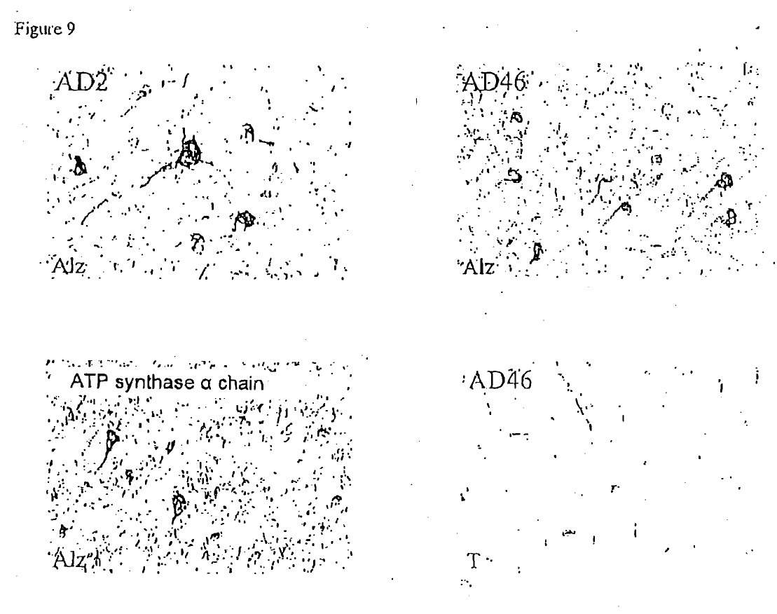

FIG. 9 illustrates an immunomarking similar to the neurofibrillary degeneration, on a section of Alzheimer's tissue, with an anti-tau antibody (AD2), AD46, and an ATP synthase α anti-chain.

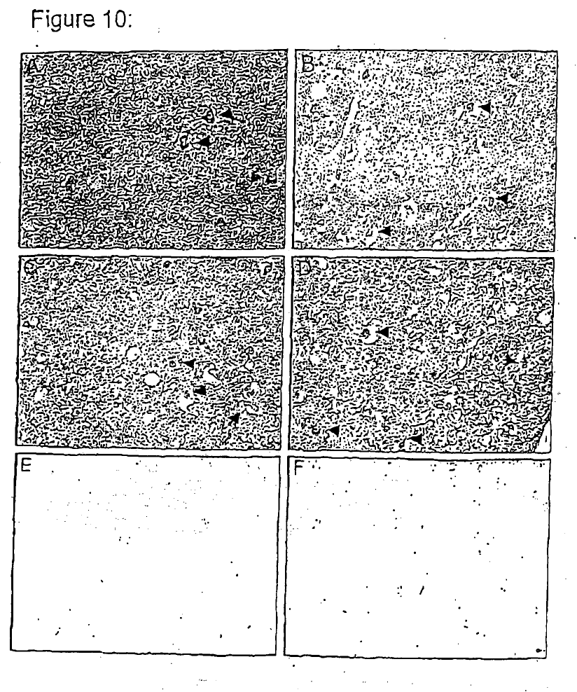

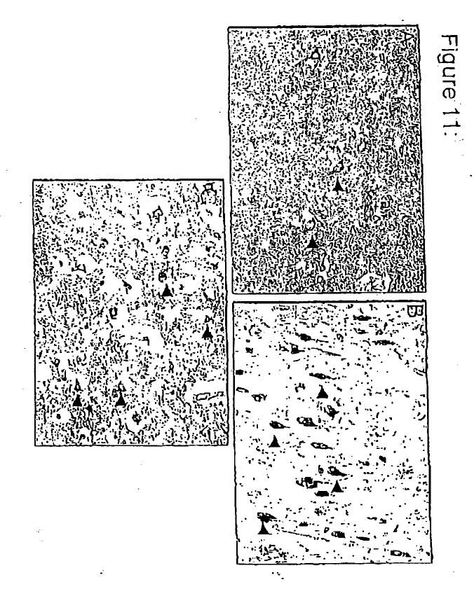

FIGS. 10 and 11 relate to the specificity of the detection of the neurofibrillary degeneration.

### EXAMPLE 1

### Demonstration of the Involvement of the ATP Synthase α Chain in Alzhoimer's Disease (AD)

A. Material and Methods

Monoclonal Antibody AD46

The monoclonal antibody AD46 was obtained using an in vitro immunization kit (Immune System, Bristol, UK) from the substance most insoluble in formic acid, of the cerebral tissue of a patient suffering with Alzheimer's disease. The immunogen was prepared following the method described by Permanne et al., 1995. The in vitro immunization kit was used following the manufacturer's recommendations. The selection of the clone was carried out by immunohistochemical study of sections of cerebral tissue from patients suffering with Alzheimer's disease.

Immunohistochemical Study and Electron Microscopy

The immunohistochemical study is carried out starting with fixed and frozen human cerebral tissue. The 15 mm sections are left to thaw for 15 minutes. The endogenous peroxidase activity is neutralized by treatment with hydrogen peroxide (1% solution) followed by several rinses in water. The sections are then equilibrated in the incubation buffer of the antibody (PBS, Sigma). The antibody is used at a final dilution of 1/1000 in PBS and incubation on the section is carried out for 2 hours at ambient temperature. After several rinses in the incubation buffer, the section is incubated with the secondary antibody coupled with the peroxidase (horseradish peroxidase conjugate anti-mouse antibodies, Sigma). The complexes are developed with a development kit (FastDAB, Sigma) following the manfacturer's instructions. The electron microscopy protocol is identical to that described by Reig et al., 1995.

Preparation of the Samples of Human Cerebral Tissue

Samples of cerebral tissue, obtained after autopsy or biopsy and stored at −80° C., were dissected with reference to an anatomical atlas, then homogenized using a Teflor® potter; in 10 volumes of 1-D lysis buffer (50 mM Tris-HCl pH 6.8; 4 mM EDTA; 5% (w/v) of SDS, 10% (v/v) of glycerol, 2% (v/v) of β-mercaptoethanol and 0.05% bromophenol blue). The samples were taken to 100° C. for 10 minutes and stored at −80° C. until used.

Fractionation of the Proteins of Human Cerebral Tissue

The cerebral tissue samples were homogenized according to a ratio of 1/10 (w/v) in Tris 10 mM buffer, pH 6.8, then centrifuged at 100,000 g for 1 hour at 4° C. The supernatant (S1) was retained and the pellet homogenized again in the same buffer complemented with 0.5% of Triton X-100. An additional stage of centrifugation was carried out under the same conditions. In total, six centrifugation processes were thus carried out after successive addition of 2% Triton X-100, 0.5% SDS, 1% SDS and 2% SDS.

According to the biochemical technique used, the corresponding buffers are added to the supernatants, at the time of use: 1 volume of 1-D lysis buffer (100 mM Tris-HCl pH 6.8; 8 mM EDTA; 10% (w/v) of SDS; 20% (v/v) of glycerol; 4% (v/v) 7-mercaptoethanol and 0.1% bromophenol blue) for a one-dimensional electrophoresis study or 1 volume of 2-D lysis buffer (7M urea; 2M thiourea; 9% Pharmalytes (D 3-10 (w/v); 4% (v/v) Triton X-100; 20 mM dithiothreitol) for a two-dimensional electrophoresis study.

Purification of the Aggregates of Tau Proteins of the Degenerating Neurons

The experimental method used is that described by Greenberg and Davies, 1990 and modified by Goedert et al., 1992. The cerebral tissue is homogenized using a Teflon potter in a solution containing 10 m-M Tris-HCl pH 7.4, 800 mM NaCl, 1 mM EGTA and 10% sucrose. The homogenate is centrifuged at 20,000 g for 30-minutes at 4° C. The supernatant is recovered, the pellet is homogenized again following the same protocol. After centrifugation following the same parameters previously mentioned, the supernatant is recovered and mixed with the first. N-lauryl sarcosine is added to the homogenate to produce a final concentration of 1% and it is placed under gentle stirring for 1 hour at ambient temperature. After centrifugation at 100,000 g for 1 hour, the pellet containing the aggregates of tau proteins is treated with 1 volume of 1-D lysis buffer.

Immunoprecipitation

The cerebral issue is homogenized using a sintered glass potter in 10 volumes of a solution containing 10 mM Tris-HCl pH 7.4, 150 M NaCl. The homogenate is centrifuged at 27,000 g for 30 minutes at 4° C. following the procedure described by Vincent and Davies (1992). The supernatant is mixed with 10 μl of protein A/G agarose. The homogenate is centrifuged at 2000 g for 10 minutes at 4° C. 10 μl of ATP synthase α anti-chain antibody, or 5 μl of the AD46 antibody or 5 μl of the AD2 antibody is added to the supernatant and stirred overnight at 4° C. 10 μl of protein A/G agarose is added to the homogenate and the mixture is stirred for 30 minutes at 4° C. A series of 3 washings is carried out. The homogenate is centrifuged at 2,000 g for 1D minutes at 4° C., 100 μl of starting buffer are added, the mixture is agitated for 5 minutes at ambient temperature and centrifuged again. This stage is repeated twice. The spheres are then incubated with 10 volumes of 1-D lysis buffer and placed at 60° C. for 5 minutes. The supernatant is used for subsequent stages of analysis.

1-D or SDS-PAGE Electrophoresis (Sodium Dodecyl Sulphate-Polyacrylamide Gel Electrophoresis)

The experiments were carried out using the Protean Xi Cell system (Biorad) according to the manufacturer's instructions. The SDS-PAGE processes were carried out according to the protocols described by Laemmli (1970) for the production of the gel. This is gel with a polyacrylamide gradient comprised between 8 and 15%. 100 μg of proteins are deposited in each track.

2-D Electrophoresis

1^{st} Dimension

The first dimension is carried out following the protocol described by O'Farrell et al., 1977. The equipment used for isoelectric focussing is the IEF Protean Cell system (Biorad) according to the manufacturer's instructions. The gel contains 9.5 M urea, 4% Triton X-100 and 4% Pharmalytes 3-10. Polymerization is initiated by the addition of ammonium persulphate and TEMED. The gel is poured into 20 cm tubes with an internal diameter of 2 mm. 50 μl of 2D homogenate are deposited on the anode side of the gel and isoelectric focussing is carried out by the application of a voltage of 400 volts over 6 hours.

2^{nd} Dimension

Before use, the gels are equilibrated for 30 minutes in an SOS-PAGE buffer (50 mM Tris pH 6.8; 10% glycerol; 2% β-mercaptoethanol; 2% SDS; 0, 05% bromophenol blue) then deposited on top of an SDS-PAGE separation gel with a polyacrylamide gradient of 8-15%.

Transfer to Nitrocellulose Membrane and Immunodot

The transfer was carried out using the Pharmacia LKB multiphor® semi-dry transfer system following she manufacturer's instructions (Amersham-Pharmacia Biotech). The proteins were transferred at 0.8 mA/cm² to a Hybond® ECL nitrocellulose membrane (Pharmacia-Amersham).

The membrane is incubated for 60 minutes in solution containing 15 mM of Tris pH 8.0, 150 mM NaCl, 0.5% Tween®-20 and 5% skimmed milk then washed with the same buffer, free frog milk and containing 0.1% of Tween-20 instead of 0.5%. The membrane is incubated, for 2 hours at ambient temperature or overnight at 4° C., with the monoclonal antibody AD46 at 1/2000th final in an incubation buffer (15 mM Tris pH 8.0, 150 mM NaCl, 0.1% Tween-20 and 4% skimmed milk).

The membrane is washed 3 times for 10 minutes in the milk-free incubation buffer.

The membrane is then incubated for 1 hour at ambient temperatures with an goat anti-mouse immunoglobulin coupled to horseradish peroxidase, at a final dilution of 1/4000th (v/v) in milk-free incubation buffer. The membrane is washed 3 times in incubation buffer and the immunoreactive polypeptides are developed using the ECL chemiluminescence kit (Pharmacia-Amersham) according to the manufacturer's instructions.

Mass Spectrometry

After 2D electrophoresis, staining of the gel is carried out. The gel is fixed for 30 minutes in a solution containing 10% acetic acid, 50% ethanol then stained in the same solution containing 0.5% Coomassie Blue R25C. Discolouration is carried out in a solution containing 30% ethanol and 5% acetic acid until a light background is obtained. The stains recognized by the monoclonal antibody AD46 are cut out of the gel and treated following the protocol described by Andersen and Mann in 2000. Mass spectrometry is carried out at the Centre Commun de Spectrometrie de Mass of the University of Lille 1.

B. Results

Immunohistochemical Study and Electron Microscopy

AD2 is an anti-tau antibody directed against a double phosphorylation site on the tau protein, which serves as a reference for studying neurofibrillary degeneration in AD (FIG. 1: AD2, Alz). The monoclonal antibody AD46 detects the neurons in neurofibrillary degeneration of the cerebral tissue of an Alzheimer's patient (FIG. 1: AD46, Alz). No neuronal marking is observed in the cerebral tissue of a control subject (FIG. 1: AD2 and AD46, T). At the electron microscopy scale, the neurons in neurofibrillary degeneration are characterized by an accumulation of pathological filaments. These filaments are constituted by aggregated tau proteins and called PHFs (for Paired. Helical Filament). They are also narrowly and specifically detected by the AD46 antibody (FIG. 2). The AD46 antibody therefore reacts with one or more essential constituents of the pathological filaments of the neurons in neurofibrillary degeneration (FIG. 2).

Biochemical Analysis of the Antigens Recognized by the Monoclonal Antibody AD46

The monoclonal antibody AD46 detects 3 bands after immunodots annotated A, B and C with an apparent molecular mass of 55.47 and 42 kDa (FIG. 3A: 1D track). After 2D electrophoresis, 4 major spots are marked (FIG. 3: part 2D). A single spot corresponds to A, with an isoelectric point of 8.2. Two spots are displayed for B, with isoelectric points 5.0 and 7.0. A single spot is displayed for C with an isoelectric point 5.8 (FIG. 3: part 2D).

Characterization of the Antigens Recognized by AD46

Each protein recognized by AD46 was isolated by 2D electrophoresis. Enzymatic digestion by trypsin was carried out in the gel and the peptide fragments recovered were analyzed by mass spectrometry.

The identification is summarized in the following table.

| Identity of | | | MM/pI | MM/pI | |
|---|---|---|---|---|---|
| the proteins | Organism | Ref. | theo. | obs. | Id. |
| C: cytoplasmic | Human | P02570 | 41.6/5.29 | 42/5.5 | MPF/WB |
| actin | | | | | |
| B: γ-enolase | Human | P09104 | 47.1/4.94 | 47/5.0 | MPF/WB |
| (EC 4.2.1.11) | | | | | |
| B: α-enolase | Human | P06733 | 47.0/6.99 | 47/7.0 | MPF |
| (EC 4.2.1.11) | | | | | |
| A: ATF synthase | Human | P25705 | 55.2/8.28 | 55/8.2 | MPF/WB |
| α chain | | | | | |
| (EC 3.6.1.34) | | | | | |

Id.: Identity of the proteins by confirmed by mass spectrometry (MPF) and immunodot (WB).
Ref.: reference of the proteins following the nomenclature of the site http://www.expasy. ch/.

The molecular masses of the fragments of each protein are used in order to interrogate the Internet databases: (MS-FIT: http://falcon.ludwig.ucl.ac.uk/ucsfhtml3.2/msfit.htm)

The results of identification as well as the identification score are summarized in the table (FIG. 4). Thus, protein A corresponds to the ATP synthase a chain, proteins B are respectively gamma enolase and alpha enolase and protein C corresponds to the cytoplasmic actin. There is no similarity of sequence between these proteins. It should however be noted that the monoclonal antibody AD46 has the strongest affinity for the 55 kDa protein, the ATP synthase a chain.

Validation of the Characterization of the 55 kDa Protein as ATP Synthase α Chain

A polyclonal antibody directed against the ATP synthase a chain was used after 2D electrophoresis (FIG. 5: Polyclonal). The ATP synthase α chain is detected at an apparent molecular mass (MM) of 55 kDa and an isoelectric point of 8.2. The AD46 antibody also recognizes the same protein; it is the ATP synthase α chain (FIG. 5: AD46).

Change of Solubility of the ATP Synthase α Chain in AD

The proteins of the cerebral tissue were dissociated following an increasing solubility gradient (cf. material and methods).

The most soluble proteins are in the Tris fraction (FIG. 6: 2nd track of the immunodots) and the increasingly less soluble proteins are recovered using detergent such as Triton X-100 (3rd and 4th track of the immunodots (FIG. 6). The proteins B and C are not modified in AD (Control, subclinical Alzheimer's, confirmed Alzheimer's immunodots, Arrows B and C). Conversely, the protein A disappears from the 0.5% Triton X-100 fraction at the subclinical stage of AD and in the 0.5% and 2% Triton-X100 fractions in confirmed Alzheimer's (Subclinical Alzheimer's and confirmed Alzheirner's immunodots, arrow A) (FIG. 6). There is therefore an early loss of solubility of the ATP synthase a chain in AD.

Confirmation of the Disappearance and of the Change in Solubility of the ATP Synthase α Chain in AD

Following the same methodology as in FIG. 6, the ATP synthase α chain is detected using a polyclonal antibody directed against the ATP synthase α chain (FIG. 7: indicated by an arrow: A (55 kDa)). In the total homogenates of cerebral tissue of a control subject and an Alzheimer's patient, the ATP synthase a cha-n is detected at 55 kDa of apparent molecular mass. It is completely absent from the Tris and 2% SDS protein fractions. It is detected in the C.5 and 2% Triton fractions and in the 0.1% SDS fraction but not in the 1% SDS fraction, of the control subject. In the protein fractions of the Alzeimer's patient, it is absent from the 0.5% Triton fraction, weakly detected in the 2% Triton fraction, and detected in the 0.1 and 1% SDS fractions. In conclusion, there is a reduction in the quantity and an insolubilization of the ATP synthase α chain in AD.

Association of Modifications of the Biochemical Properties of the ATP Synthase α Chain with the Neurofibrillary Degeneration Process

These changes in biochemical properties are associated with the process of neurofibrillary degeneration of AD. In fact, co-marking with the AD2 monoclonal antibody was carried out in the same experiment. The pathological tau proteins (FIG. 7: annotated Tau 60, 64, 69) are detected only in the Alzheizrer's patient fractions and not at all in the control subject fractions (FIG. 7). It should be noted that outside the total homogenate, the protein fraction which contains the largest quantity of pathological tau protein corresponds to the 1% SDS fraction (FIG. 7). This fraction also corresponds to that where the ATP synthase a chair is found in AD and not in the control subject. A relationship therefore exists between the change in solubility of the ATP synthase α chain and the presence of pathological tau proteins which are the biochemical signature of neurofibrillary degeneration in AD.

Copurification of the ATP Synthase a chain with the Aggregates of Tau Proteins and Co-Immunoprecipitation of the Aggregated Proteins with the ATP Synthase α Chain

The aggregated tau proteins are purified in the sarkosyl-insoluble fraction and not at all the tau proteins of the control individual (FIG. 5A: 100K P fraction, Control and Alz). The polyclonal antibody directed against the ATP synthase α chain detects the ATP synthase α chain in the sarkosyl-insoluble fraction of the Alzheimer's patient only (FIG. 8A: 100K P fraction, polyclonal anti-ATP synthase α chain marking). This result demonstrates the copurification of the ATP synthase α chain with the aggregates of tau proteins of the neurons in neurofibrillary degeneration and therefore the direct association of the ATP synthase α chain with the aggregative process of the tau proteins.

This result is backed up by the immunoprecipitation experiments (FIG. 8B). The immunoprecipitation of the ATP synthase α chain using the AD46 antibody or of the polyclonal anti-ATP synthase α chain reveals the presence of hyperphosphorylated tau proteins, which are demonstrated, by marking with the monoclonal antibody AD2 or a polyclonal antibody directed against the carboxy-terminal region of the tau protein (FIG. 8B: the two Alz tracks). It should be noted that the tau proteins of the cerebral tissue of a control individual are not displayed following the same experimental procedure (FIG. 8B: the two Control subject tracks). This demonstrates the specificity of the association of the ATP synthase α chain with the abnormally phosphorylated proteins, the basic constituents of the aggregates, and not at all with the normal tau proteins. The association of the ATP synthase α chain with the aggregated tau proteins is therefore to be linked directly to the physiopathological process and not an intrinsic affinity of the ATP synthase α chain for the tau proteins.

Confirmation of the Involvement of the ATP Synthase α Chain in the Neurofibrillary Degeneration Process in AD

The neurons in neurofibrillary degeneration are detected by the monoclonal antibodies AD2 and AD46 on cerebral tissue of AD patients. Normal human cerebral tissue is not marked by the AD46 antibody. A polyclonal antibody directed against the ATP synthase α chain detects the neurons in neurofibrillary degeneration of the cerebral tissue of AD patients (FIG. 9). Conversely, antibodies directed against the enolases or cytoplasmic actin do not allow selective detection of the neurons in neurofibrillary degeneration in AD.

The above results show the existence of a specific accumulation of the ATP synthase α chain, associated with early changes in the biochemical properties of this protein in Alzheimer's disease. The ATP synthase α chain is therefore directly involved in the process of neurofibrillary degeneration in AD, and it therefore constitutes a novel diagnostic and therapeutic target.

### EXAMPLE 2

### Detection of Neurofibrillary Degeneration

I. The Detection of Neurofibrillary Degeneration is Carried Out Using Antibodies Directed Against the ATP-Synthase α Chain and by Immunomarking with Antibodies Directed Against Other Mitochondrial Complex V Proteins.

A series of sections of the temporal cortex of a patient suffering with Alzheimer's disease were used in order to carry out immunohistochemical analysis. The neurons in neurofibrillary degeneration are indicated by arrows.

FIG. 10 presents the following experiments:

(A) Detection of neurofibrillary degeneration using the monoclonal antibody AD2, directed against the phosphorylated tau proteins.

ATP Synthase α Chain:

(B) Detection of neurofibrillary degeneration using the monoclonal antibody AD46.

(C) Detection of neurofibrillary degeneration using a polyclonal antibody directed against the amino-terminal part of the ATP synthase α chain. The polyclonal antibody used was prepared from the following peptide (SEQ ID No. 1) corresponding to the amino-terminal region of the ATP synthase α chain which starts at amino acid 45 and ends at amino acid 58: CKTGTAEMSSILEER

(D) Detection of neurofibrillary degeneration using a polyclonal antibody directed against the carboxy-terminal part of the ATP synthase α chain. The polyclonal antibody used was prepared from the following peptide (SEQ ID No. 2) corresponding to the carboxy-terminal region of the ATP synthase α chain which starts at amino acid 540 and ends at the last amino acid 553: CLKEIVTNFLAGFEA

The peptides of experiments C and D were synthesized by Neosystem (Strasbourg, France) and a cysteine (C) was added to their amino-terminal end in order to allow coupling to the carrier which is ovalcumin. The polyclonal antibodies were then produced by Neosystem, in rabbits.

Other Mitochondrial Complex V Proteins:

(E) Immunomarking carried out using a monoclonal antibody directed against the ATP synthase beta chain.

(F) Immunomarking carried out using a monoclonal antibody directed against the OSCP (Oligomycin Sensitivity-Conferring Protein).

The monoclonal antibodies of experiments E and F were obtained from Molecular Probes (Leiden, Netherlands).

Results:

This figure shows the specificity of the association of the ATP synthase α chain with the neurofibrillary degeneration process.

In fact, two polyclonal antibodies directed against the amino-terminal end and the carboxy-terminal end of the ATP synthase α chain were produced. These two antibodies mark the neurons in neurofibrillary degeneration (NFD) in the cerebral tissue of a patient suffering from Alzheimer's disease. The marking is completely superimposable on that obtained with the AD46 antibody described in Example 1.

Conversely, the use of monoclonal ant-bodies directed against other mitochondrial complex V proteins, a complex to which the α chain also belongs, does not mark the neurons in NFD. This result therefore shows the specificity of the association of the ATP synthase α chain with the NFD process an Alzheimer's disease.

II. Detection of neurofibrillary degeneration was also carried out using a commercial anti-ATP synthase α chain antibody. Moreover, the antibodies according to the invention were tested in order to demonstrate their capacity to detect neurofibrillary degeneration in other neurodegenerative diseases.

FIG. 11 presents the following experiments:

ATP Synthase α Chain:

(A) Detection of neurofibrillary degeneration using a monoclonal antibody directed against the ATP synthase a chain (Molecular Probes, Leaden, Netherlands).

Other Neurodegenerative Diseases:

(B) Detection of neurofibrillary degeneration in the frontal cortex of a patient suffering from Pick's disease using the monoclonal antibody AD46. Pick bodies which are the intraneuronal neuropathological structures characteristic of this pathology are indicated by arrows.

(C) Detection of neurofibrillary degeneration in the frontal cortex of a patient suffering from Down's syndrome (Trisomy 21) using the monoclonal antibody AD46. The neurons in neurofibrillary degeneration are indicated by arrows.

Result:

This last figure shows that the monoclonal antibody AD46 also detects the neurons in NFD of other neurodegenerative diseases. Pick's disease and Trisomy 21 are shown as examples. The ATP synthase α chain is therefore also involved in the NFD process due to other neurodegenerative diseases.

### EXAMPLE 3

### Method for the Preparation of Immunological Tools Against the Pathological ATP Synthase α Chain, in Particular Against the Pathological Epitopes

The method can comprise the following stages:

Selective extraction of neurofibrillary degeneration from human tissue affected by the Alzheimer's type degenerative process, using increasingly powerful solubilization buffers (tris, triton, SDS, sarkosyl, formic acid).

Extraction of the pathological ATP synthase α chain linked to the tau proteins of neurofibrillary degeneration by an immunological and biochemical approach (for example, immunoprecipitation of the pathological ATP synthase α chain by a polyclonal antibody or by AD46, then purification of the pathological ATP synthase α chain of the product immunoprecipitated by electrophoresis, HPLC, or any other appropriate method).

Use of the purified pathological ATP synthase α chain as antigen for the production of polyclonal or monoclonal immunological tools. Use of the complete molecule as epitope or controlled digestion of the purified pathological ATP synthase α chain, determination of the pathological epitope by immunopurification using the AD46 antibody and use of the epitope for the production of polyclonal or monoclonal antibodies.

For the production of monoclonal antibodies, selection of the hybridomas obtained for specific assays of the normal and pathological epitopes of the pathological ATP synthase a chain.

Ideally, the clones selected should possess the following immunological properties:
1. Detection of the Alzheimer's-type neurodegenerative process by immunohistochemical study
2. Detection of the pathological ATP synthase α chain
3. Co-immunoprecipitation of the pathological tau proteins.

### REFERENCES

Andersen J S, Mann M (2000) Functional genomics by mass spectrometry. FEBS Letters 480, 25-31.
Delacourte A, David J P, Sergeant N, Buee L, Wattez A, Vermersch P, et al. (1999) The biochemical pathway of neurofibrillary degeneration in aging and Alzheimer's disease. Neurology, 52, 1:58-65.
Greenberg, S. G. and P. Davies (1990). “A preparation of Alzheimer paired, helical filaments that displays distinct tau proteins by polyacrylamide gel electrophoresis.” Proc Natl Acad Sci USA 87 (15): 5827-31.
Goedert, M., M. G. Spillantini, N. J. Cairns and R. A. Crowther (1992). “Tau proteins of Alzheimer paired helical filaments: abnormal phosphorylation of all six brain isoforms.” Neuron 8(1): 159-68.
Laemmli U K (1970) Cleavage of structural proteins during the assembly of the head of bacteriophage T4. Nature, 227, 660-5.
O'Farrell, P Z, Goodman H M, et al. (1977a). “High resolution two-dimensional electrophoresis of basic as well as acidic proteins.” Cell 12 (4), 1133-41.
O'Farrell, P. H. and P. Z. O'Farrell (1977b). “Two-dimensional polyacrylamide gel electrophoretic fractionation.” Methods Cell Biol 16, 407-20.

Permanne B, Buee L, David J P, Fallet-BiancoC, Di Menza C, Delacourte A (1995) Quantitation of Alzheimer's amyloid peptide and Identification of related amyloid proteins by dot-blot immunoassay. Brain Res, 685,134-62.
Reig S, Buee-Scherrer v, Mourton-Gilles C, Defossez A, Delacourte A, Beauvillain J C, et al. (1995) Immunogold labelling of paired helical filaments and amyloid fibrils by specific monoclonal and polyclonal antibodies. Acta Neuropathol, 90, 441-7.
Vincent, I. J. and P. Davies (1992). “A protein kinase associated with paired helical filaments in Alzheimer disease.” Proc Natl Acad Sci USA 89 (7): 2878-82.

1. Markers of the neurodegenerative process, constituted by the ATP synthase α chain having undergone pathological modifications resulting from said process.

2. Markers according to claim 1, wherein the modifications of the ATP synthase α chain are of functional, location, structural and/or antigenic type.

3. Markers according to claim 1, wherein the neurodegenerative process is that of any pathology with a neurofibrillary degeneration process and aggregation of tau proteins, in particular, that of Alzeheimer's disease.

4. Markers according to claim 3, wherein one of the functional modifications of the ATP synthase α chain is its insolubility.

5. Markers according to claim 3, wherein one of the location modifications of the ATP synthase α chain is its location in the cytoplasm of the cell.

6. Markers according to claim 3, wherein one of the structural modifications of the ATP synthase α chain is the formation of aggregates at the level of the cerebrum.

7. Markers according to claim 1, wherein they interact with the tau proteins.

8. Method of detection and/or of diagnosis in vitro of the neurodegenerative process, characterized in that wherein one of the markers according to claim 1 is detected in a sample to be analyzed.

9. Method according to claim 8, wherein it comprises the use of sets of antibodies directed against the normal protein and/or against modifications of the ATP synthase α chain.

10. Method according to claim 8, wherein it is used for detection of the degenerative process of any pathology with a neurofibrillary degeneration process and aggregation of tau proteins, in particular that of Alzheimer's disease.

11. Method according to claim 8, wherein immuno-chemical detection is used, in particular by 1D and/or 2D electrophoresis coupled with an immunodot, development by polyclonal antibodies or monoclonal antibodies directed against the ATP synthase a chain, immuno-assay and/or radioimmuno-assay, optionally completed by mass spectrometry analysis.

12. Method according to claim 8, wherein the samples to be analyzed used in said method include neuronal tissues or cells, non-neuronal tissues or cells, in particular bilogical liquids, preferably blood.

13. Diagnostic method according to claim 8, wherein the degree of pathology is moreover evaluated by establishing an index based on the relationship between the normal level of the ATP synthase α chains in control subjects in a defined protein fraction, with respect to the level observed at an advanced stage of Alzheimer's disease.

14. Diagnostic method according to claim 8, wherein the degree of pathology is moreover evaluated by establishing an index based on modifications of the ATP synthase α chain in a patient compared with a control subject.

15. Uses of the method according to claim 8, wherein for assisting with ante and post-mortem diagnosis of the neurodegenerative diseases, in particular Alzheimer's disease, at the subclinical stage.

16. Animal or cell model, wherein it expresses an ATP synthase α chain having a maturation signal defect or a post-translational modification anomaly.

17. Use of the method according to claim 8, wherein for pharmacological screening and therapeutic tests on molecules effective against the neurodegenerative pathologies, in particular of Alzheimer's disease type.

18. Use of the method according to claim 8, wherein in order to establish and validate cell models and/or animal models of neurodegenerative pathologies, in particular of Alzheimer's disease.

19. Use of a kit for the detection of the ATP synthase α chain, for the diagnosis of neurodegenerative diseases, in particular for the detection of Alzheimer's disease.

20. Polyclonal and/or monoclonal antibodies directed against patterns of pathological conformation of the ATP synthase α chain resulting from a neurodegenerative process.

21. Diagnostic kit characterized in that it comprises sets of antibodies according to claim 20.

22. Diagnostic kit according to claim 21, wherein said kit contains reagents making it possible to carry out an immunochemical assay, in particular of ELISA, imunodot, Western blots, dots-blots, radioimmuno-assay or immuno-assay type.
